(19) United States
(12) Patent Application Publication
(10) Pub. No.: US 2021/0298690 A1
(43) Pub. Date: Sep. 30, 2021

(54) SYSTEM AND METHOD FOR COMMUNICATING HEALTH-RELATED MESSAGES REGARDING VENTILATED PATIENTS

(71) Applicant: Vyaire Medical, Inc., Mettawa, IL (US)

(72) Inventors: Christopher M. VARGA, Laguna Hills, CA (US); Hasnain SOMJI, Irvine, CA (US)

(21) Appl. No.: 16/828,906

(22) Filed: Mar. 24, 2020

(51) Int. Cl. A61B 5/00; A61B 5/08; G16H 10/60; G16H 40/67; G16H 50/50

(52) U.S. Cl. CPC A61B 5/743; A61B 5/72; G16H 50/50; G16H 10/60; G16H 40/67; A61B 5/0803

(57) ABSTRACT

The disclosed system receives, from a plurality of ventilators, ventilation data including physiological statistics for a plurality of patients currently receiving ventilation from the plurality of ventilators, and provides, to a plurality of user devices, a user interface configured to present the plurality of patients for selection by a respective user, receive from the respective user a selection of one or more selected patients of the plurality of patients, and provide, to the respective user, messages relating to physiological measurements of the selected patients when the physiological measurements of the selected patients meet predetermined criteria. Responsive to a first physiological measurement of a ventilated patient satisfying a predetermined threshold or range of values, a message is sent by the system to a user device for display to a user authenticated to the user interface, the message including information pertaining to the first physiological measurement.

### BACKGROUND FIELD

The subject technology addresses deficiencies commonly encountered in hospital care and medical care user and system interfaces with regard to the communication of health-related information for ventilated patients.

### SUMMARY

Systems and/or strategies for determining priority of patient care (e.g. who to see when and whom to see first) are generally rudimentary, involving isolated human interpretation of equipment/device alarms or time-based patient spot checks. Visual systems for determining status of patients are insufficient and noisy because of the overwhelming volume of information which must be stitched together to determine a clinical action. The ability to determine status of a patient population may be further exacerbated due to an unexpected influx of patients who are in need of similar critical care systems, such as ventilation.

Clinicians such as therapists, nurses and doctors in the healthcare environment today frequently carry mobile phones (e.g. smart phones) or other smart devices (e.g. tablets). Alternatively clinicians may have computers on wheels which are carted around with them or kept in patient areas (e.g. ICUs). Current software and digital applications which reside on these devices for patient monitoring or patient care are often as complex as the devices they are mirroring. For example, a patient vital signs monitoring device which measures heart rate, SpO2, blood pressure, temperature and end-tidal CO2 may contain a large amount of numbers, indices, graphs and data elements which can be overwhelming. A mobile application which provides this same data remotely on a smaller screen may provide some convenience but is equally overwhelming in terms of interpreting data and deciding based on this data what to do with a patient. As another example, a mechanical ventilator contains significantly more data elements, waveforms, trends and information than a patient vitals monitor and provides an even more overwhelming scenario for data interpretation which can be exacerbated if constrained or confined to a mobile environment (i.e. smaller screen or form factor). Listening to alarms or transmitting alarms to mobile devices is equally overwhelming because it does not solve the fundamental audible and visual overload which exists. What is needed is a simplification of care in the noisy healthcare environment which can provide for mobile, visual, actionable insights that enable prioritization, customization and coordination of patient care as well as management of process indicators, equipment maintenance, order management and personnel. Accordingly, the subject technology addresses deficiencies in the current environment for mobile-based clinical insights or the lack thereof.

The subject technology addresses deficiencies commonly encountered in hospital care and medical care involving interpretation of health-related information for ventilated patients by, in part, producing actionable clinical insights and disseminating said actionable clinical insights to caregivers. The subject technology further addresses similar deficiencies in interpretation of data and dissemination of insights related to hospital equipment and devices, cooperation and coordination of care between adjacent clinical specialties (e.g. respiratory therapy, nursing, physicians), time-sensitive patient-care tasks, and quality and process metrics relevant to hospital performance.

According to various implementations, the subject technology includes a communication system configured to communicate health-related information pertaining to ventilated patients. The disclosed communication system includes one or more processors and a memory. The memory includes instructions stored thereon that, when executed by the one or more processors, cause the one or more processors to perform operations for performing a method of communicating health-related messages regarding ventilated patients. The method includes receiving, from a plurality of ventilators, ventilation data including physiological measurements for a plurality of patients currently receiving ventilation from the plurality of ventilators; providing, to a plurality of user devices, a user interface configured to present the plurality of patients for selection by a respective user, receive from the respective user a selection of one or more selected patients of the plurality of patients, and provide, to the respective user, messages relating to the physiological measurements of the selected patients when the physiological measurements of the selected patients meet predetermined criteria; receiving, from a first instance of the user interface operating on a first user device of the plurality of user devices, an indication that a first user authenticated to the user interface selected a first ventilated patient from the plurality of patients; determining, from the received ventilation data, that a first physiological measurement associated with the first ventilated patient satisfies a predetermined threshold or range of values; and responsive to the first physiological measurement of the first ventilated patient satisfying the predetermined threshold or range of values, sending a message pertaining to the first physiological measurement to a the first user device for display by the first instance of the user interface when the first user is authenticated to the first user interface. Other aspects include corresponding systems, apparatuses, and computer program products for implementation of the computer-implemented method.

Further aspects of the subject technology, features, and advantages, as well as the structure and operation of various aspects of the subject technology are described in detail below with reference to accompanying drawings.

### DESCRIPTION OF THE FIGURES

Various objects, features, and advantages of the present disclosure can be more fully appreciated with reference to the following detailed description when considered in connection with the following drawings, in which like reference numerals identify like elements. The following drawings are for the purpose of illustration only and are not intended to be limiting of this disclosure, the scope of which is set forth in the claims that follow.

FIG. 1 depicts an example sign-in or log-in screen for the disclosed messaging system, displayed on a mobile device, according to various aspects of the subject technology.

FIG. 2 depicts an example patient selection screen, according to various aspects of the subject technology.

FIG. 3 depicts an example user interface displaying a lock screen, including a display of pushed clinical insight messages, according to various aspects of the subject technology.

FIG. 4 depicts a first example display screen for a selected patient, including a toggled layout design by which a user can move between active messages and all (logged) messages for a patient, according to various aspects of the subject technology.

FIG. 5 depicts a second example display screen for a selected patient, including a toggled layout design, with the ‘All’ message category displayed, according to various aspects of the subject technology.

FIG. 6 depicts an example menu dialog, according to various aspects of the subject technology.

FIG. 7 depicts an example user interface for showing those patients which are subscribed to by the currently logged in user, according to various aspects of the subject technology.

FIG. 8 depicts an example display screen including information pertaining to the Ventilator Length of Stay (in days) for a patient population at a hospital or within a care area (e.g., associated with the user), according aspects of the subject technology.

FIG. 9 depicts an example display screen indicating patient trends with regard to oxygen concentration and lung pressure, according to various aspects of the subject technology.

FIG. 10 is an example display screen indicating the health of ventilation-related equipment, according to various aspects of the subject technology.

FIG. 11 depicts an example display screen indicating the status of a selected healthcare device, according to various aspects of the subject technology.

FIG. 12 depicts an example display screen indicating patients watched by the logged in user, with additional alert indicators for patients whose information includes a time-sensitive component or content, according to various aspects of the subject technology.

FIG. 13 depicts an example patient selection screen, with patient information displayed in a pop-out dialog, according to various aspects of the subject technology.

FIG. 14 depicts an example display screen for a selected patient, including a toggled layout design, with patient information displayed in a pop-out dialog, according to various aspects of the subject technology.

FIG. 15 depicts an example display screen indicating patients watched by the logged in user, with patient information displayed in a pop-out dialog, according to various aspects of the subject technology.

FIG. 16 is a block diagram depicting a system for communicating health-related messages regarding ventilated patients, including an example ventilation device and a ventilation management system, according to certain aspects of the subject technology.

FIG. 17 depicts an example flow chart of a method of communicating health-related messages regarding ventilated patients, according to aspects of the subject technology.

FIG. 18 is a conceptual diagram illustrating an example electronic system for communicating health-related messages regarding ventilated patients, according to aspects of the subject technology.

### DESCRIPTION

While aspects of the subject technology are described herein with reference to illustrative examples for particular applications, it should be understood that the subject technology is not limited to those particular applications. Those skilled in the art with access to the teachings provided herein will recognize additional modifications, applications, and aspects within the scope thereof and additional fields in which the subject technology would be of significant utility.

The subject technology comprises a computer-enabled communication system, including a visual environment, which enables delivery of health-related messages to mobile devices from a cloud-based or connected analytics platform. Clinical insight messages with regard to ventilated patients are pushed to a user as notifications or messages on a screen and then logged thereafter for subsequent viewing. Users may comprise physicians, nurses, therapists, technicians, engineers, or other individuals that interact with the patients, or healthcare systems, staff, and devices. Delivery of messages through the system enables caregivers to act upon simplified insights distilled from myriad data sources, to coordinate and cooperate with other clinical staff members or users, to prioritize patient care, and to make timely decisions on what care or therapy to provide. Messages may be customized to user care areas or disciplines, user clinical profiles, user hierarchical levels and facility profiles or specifications. Messages may be related to respiratory care, sedation and drug therapy, vital signs, imaging, laboratory results, demographics, disease states, progression of condition, equipment status, and department or hospital metrics. While the term “clinical insight” may refer to information provided within a message transmitted through the system, the term “clinical insight” may be used synonymously with the term “notification” or “message” herein to describe messages transmitted through the disclosed system.

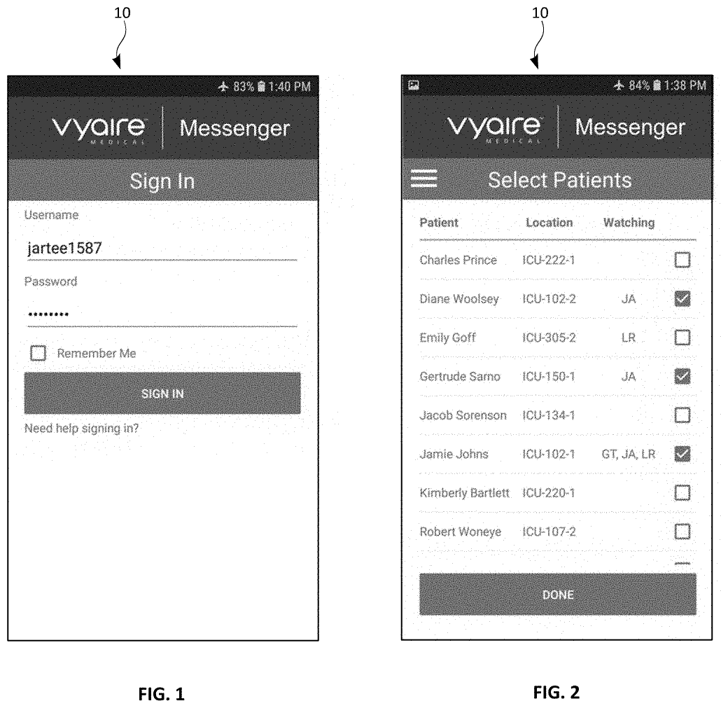

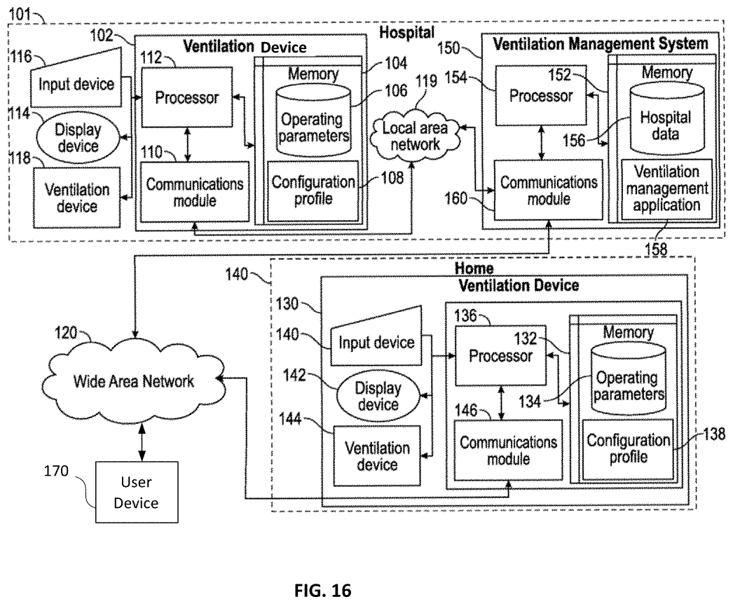

FIG. 1 depicts an example sign-in or log-in screen of a user interface 10 for the disclosed messaging system, displayed on a mobile device (e.g., a user device 170 of FIG. 16), according to various aspects of the subject technology. The user interface is configured to display various display screens disclosed herein. The log-in screen of FIG. 1 may be used when a user accesses the user interface 10 for a first time, for example, when a user starts their shift. In some implementations, the log-in screen will be displayed each time the user accesses the applications. The system requires each user to authenticate to a user interface 10 (e.g., by way of a server that provides the interface), and that each user is authorized to receive patient health data for patients operably connected to the system, or for whom the system receives the data. The system only provides information and messages pertaining to patients of the authorized user, and when consistent with patient privacy regulations. Access is typically granted when the user enters the appropriate credentials through the user interface, or by other means configured to communicate the credentials to the user interface and corresponding systems.

FIG. 2 depicts an example patient selection screen, according to various aspects of the subject technology. Upon logging or signing in, the user interface 10 may display the screen of FIG. 2 which enables an authenticated user to make selections which will determine messages they will receive. A clinical user such as a respiratory therapist is taken to the depicted screen for selecting patients. On this screen appears a list of all patients in an intensive care unit at a facility or in the collection of intensive care units (ICUs) at a facility. Facilities may contain difference kinds of ICUs such as neonatal (NICU), surgical (SICU), medical (MICU), pediatric (PICU), and more. In the depicted implementation, the screen for selecting patients contains patient names, their ICU bed location within the hospital as well as who is receiving messages for them.

Users of the system may utilize the interface to subscribe to patients admitted to or currently being cared for in a care area. The user interface 10 of FIG. 2 includes a column called “Watching” which identifies/displays the initials of users who have subscribed to receive insights on each patient. Display of the initials of users or clinicians that are watching a patient or receiving messages enables caregivers to coordinate care with other users for a particular patient and to determine quickly who is receiving messages for which patients and which patients may not be being watched by anyone. By touching and holding a finger in place on a set of initials, the application displays the full name, specialty, and any other information about the user that may beneficially identify them to other users or enable them to make clinical determinations for cooperation. A patient may be subscribed to by multiple users such that all users subscribed to a patient are receiving mobile insights for that patient. When a user selects a patient by touching their name or check box or similar selection target, the user's initials are added to the “watching” column. FIG. 2 illustrates one such example in which the user's initials are “JA”. Once the user has selected patients, they can press a ‘Done’ button to signal completion of the subscribing process (e.g., to a central server). Once the user is subscribed to one or more patients, the system is configured to transmit clinical insights (as messages or notifications) to the device on which the application is operating (e.g., on the user's mobile device or phone).

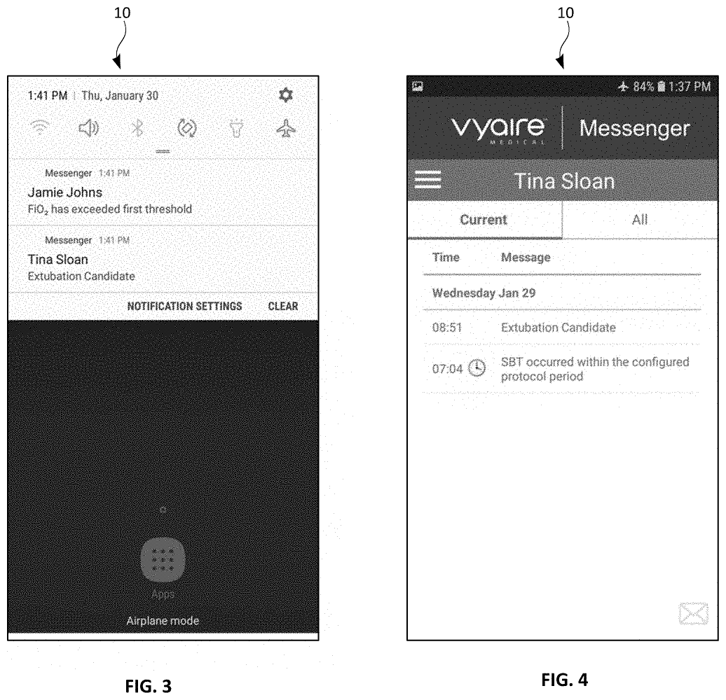

FIG. 3 depicts an example user interface 10 displaying a lock screen, including a display of pushed clinical insight messages, according to various aspects of the subject technology. In this implementation, push notifications or messages containing clinical insights may be delivered to the computing device and displayed on the lock screen while the user interface is in a locked mode.

According to some implementations, the system (e.g., a centralized server of the system) may include machine learning algorithms to look for patterns in data and project what steps should be taken with regard to patient care. Clinical insight information may be automatically generated that corresponds to clinical thresholds being reached (e.g., an oxygen level falling below a predetermined percentage), or future events or actions that should be performed. In one example, if an event X occurs, then a message may be pushed to the user device. In some implementations, the system may determine, based on X occurring in the past, that Y will occur at a future time. Depending on the event, the system may identify a future time point, signal a review of data at that specific future time point, and signal a likelihood of an event happening or signal an alert for a patient if the likelihood is a significant or morbid/undesirable event. In another example, machine learning may identify likely patient paths from a given pattern of data and, in some implementations, automatically make its own recommendations with regard to adjustments to care systems (e.g., as an autopilot). For example, on identifying a flow rate of ventilation is not providing a desired effect on the patient, an adjusted flow rate may be included in the clinical insight (e.g., within predetermined limits) to move the patient towards the desired effect. An example alert may include a text message indicating “heads up, your patient X is trending towards a ventilator-associated event” or “heads up, your patient Y is ready for weaning from ventilation.”

In the depicted example, one push notification indicates that a patient's inspired oxygen fraction has exceeded a first threshold and the other notification indicates that a patient is ready for extubation. These are actionable insights that the caregiver may react to for improved patient care and prioritization of patient care. Tapping on either push notification causes the user interface to take the user directly to a page which is dedicated to the related patient. For example, by tapping on the user named Tina Sloan, the user is taken to Tina Sloan's screen as shown in FIG. 4.

FIG. 4 depicts a first example display screen for a selected patient, including a toggled layout design by which a user can move between active messages and all (logged) messages for a patient, according to various aspects of the subject technology. Each message may be associated with date and time stamps, covering a time period which goes back to patient admission.

In the depicted example, the ‘Active’ message category is displayed. A user can see in this case two displayed active messages for patient Tina Sloan. The system is configured to automatically determine (e.g., based on predetermined conditions or learned data) when a message requires a time-sensitive action or response. The user interface of FIG. 4 includes a clock face icon present on one message to indicate that the message contains a time-sensitive component. For example, during coordination of care between nursing and respiratory therapy for a ventilated patient, spontaneous breathing trials (SBTs) and sedation awakening trials (SATs) must be coordinated beneficially for the patient. By displaying a clock icon to the users, they are alerted to a time-based message which can aid in coordination and time management.

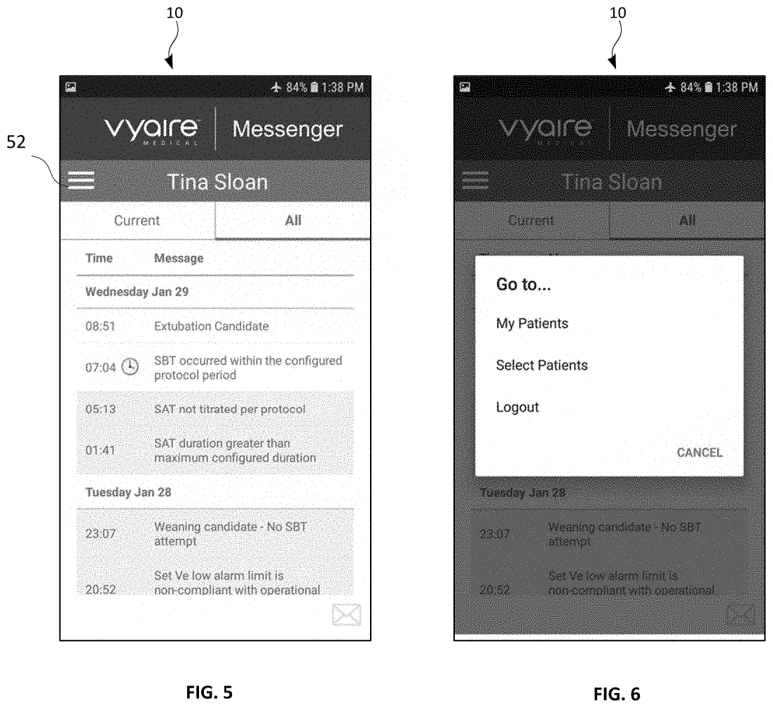

FIG. 5 depicts a second example display screen for a selected patient, including a toggled layout design, with the ‘All’ message category displayed, according to various aspects of the subject technology. In this example, the user interface 10 displays to the user all of the messages which have been generated by the analytics engine over the course of the stay of the patient at the hospital. The system logs messages by date and time to provide a historical view of the patient and to provide continuity of care between users and shifts. By displaying all logged messages for a patient history, the system enables a caregiver to determine improvements in course of action for future scenarios with the same patient or other patients. In some implementations, the user interface may include, for each message in the logged or All category, a field showing the initials of all users who were subscribed to messages for that patient at the time of the message push or generation. In this way, it is possible to both reconstruct a care scenario as well as to determine whom to speak to about past decisions or actions in the care process. In the depicted example of FIG. 5, each screen also contains an icon 52 near the top left with three horizontal lines which indicate a menu function. The user interface is configured such that, by pressing on this icon in any screen of the invention, the user is taken to a menu screen as shown in FIG. 6.

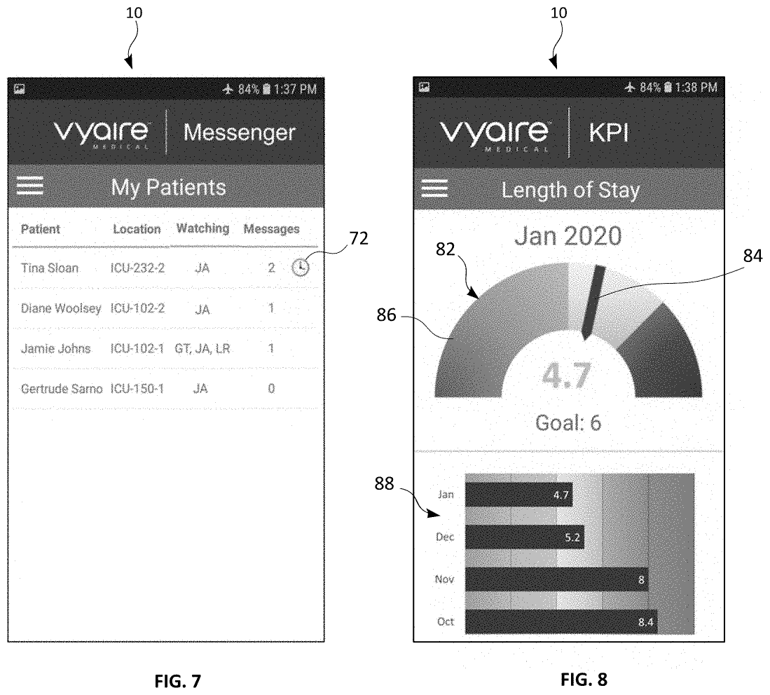

FIG. 6 depicts an example menu dialog, according to various aspects of the subject technology. On the menu, the user may choose the ‘Select Patients’ screen, a ‘My Patients’ screen, or to Logout. If the user selects the ‘My Patients’ screen, they are taken directly to a screen such as that shown in FIG. 7. This screen is also displayed directly when a user taps the ‘Done’ button on the ‘Select Patients’ screen shown earlier.

FIG. 7 depicts an example user interface for showing those patients which are subscribed to by the currently logged in user, according to various aspects of the subject technology. The ‘My Patients’ shows only the message status of the patients which the user has selected or subscribed to receive messages for. The ‘My Patients’ screen in FIG. 7 lists each patient the user is subscribed to, their location, who is receiving messages for that patient, and the number of active messages or insights that exist for each. The list of patients is sorted by the total number of messages for each patient which can help to prioritize care. For example, a caregiver or user may choose to see a patient who has the most messages and/or highest priority messages or insights delivered. Typically, the more active messages or notifications which are pushed out for a patient, the more actions there are to take and therefore this may drive priority. On the other hand, if a time-sensitive message or notification exists, the patient associated with the time-sensitive status may be give priority over a patient with a greater number of messages or insights, but nothing immediately time-sensitive. In this way, the subject technology places an icon 72 next to the messages number for any patient in the list whose messages have a time-sensitive nature or marker. In this manner, the visual identifier immediately communicates to a caregiver that time is important.

By tapping on any patient in the ‘My Patients’ list, the user may be taken directly to a display screen that includes information for that patient. In some implementations, this patient screen may be similar to FIG. 4, wherein a user taps directly on a push notification which has been received for that patient.

The system of the subject technology comprises a set of mobile accessible applications with visuals that provide instant access to information pertaining to the health of ventilated patients. In this regard, the system of the subject technology is configured to convey (e.g., via the mobile application) the health of a patient population and/or the performance of a facility with respect to both the amount of time that patients are spending on ventilators and in the ICU as well as how both individuals and patient populations are trending with respect to ventilator-associated events (VAEs). Both time spent on ventilators (also known as Ventilator Length of Stay (VLOS) and events associated with being on a ventilator such as exposure to pathogens that can lead to pneumonia and other negative conditions are factors that both deteriorate patient outcomes as well as cost hospitals significant amounts of money. Today there is no convenient method to access this information or have it at the fingertips of clinicians to assess the health of patients and a facility's performance.

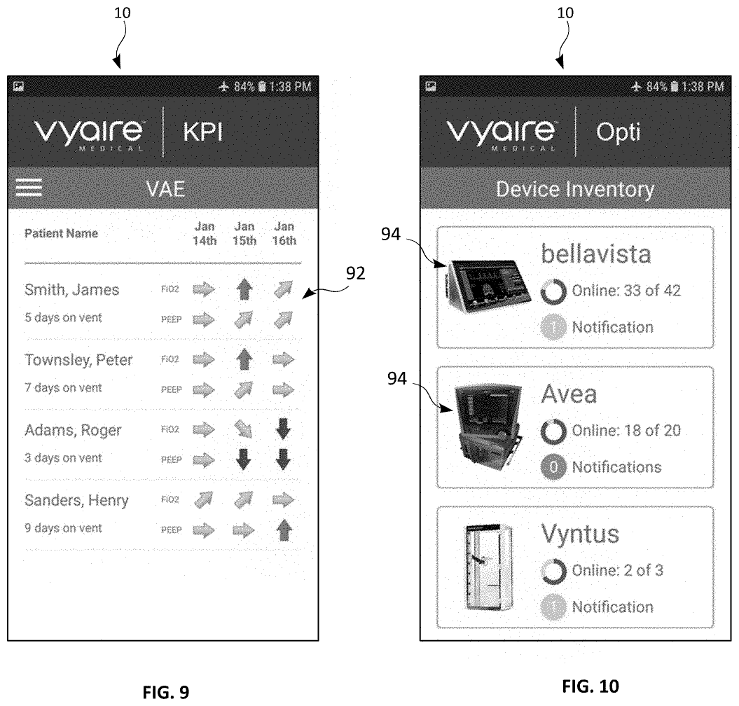

FIG. 8 depicts an example display screen including information pertaining to the ventilator length of stay (in days) for a patient population at a hospital or within a care area (e.g., associated with the user), according aspects of the subject technology. The example display conveys key metrics in a visual dial 82, with a graphical pointer 84 indicating the current length of stay by the pointer being associated with or pointing to a predetermined area of the visual dial. Each predetermined area may be color-coded (e.g., in green, red and yellow), with each color-coded area corresponding to a range of values or values meeting a predetermined threshold for the length of stay. The information presented in the example visual display also includes a target length of stay 86 (e.g., indicated by a first area color-coded in green). The display screen also depicts historical data related to length of stay performance for previous time periods, which could include months or quarters or any other time period of interest. In the depicted example, the historical data is represented by a horizontal bar chart (or graph) 88, with each horizontal bar having a length corresponding to a respective value within an associated color-coded predetermined area. Other implementations may include a vertical bar chart, or other heuristic charts. Targets and historical length of stay values may also appear in other embodiments as ticks along the dial indicator as opposed to text or bar graphs. A menu button appears on the upper left part of the screen. By tapping the menu icon, a user may select another screen called VAE as shown in FIG. 9, which provides instantaneous access to a Ventilator-Associated Event (VAE) surveillance visual.

FIG. 9 depicts an example display screen indicating patient trends with regard to oxygen concentration and lung pressure, according to various aspects of the subject technology. On this screen, patients are listed by name along with the number of days they have been on the ventilator, as well as arrows related to their levels of fraction of inspired oxygen (FiO2) and positive-end expiratory pressure (PEEP). Arrows 92 are displayed showing whether levels of these parameters over the course of days are staying the same (horizontal), increasing (diagonal up), decreasing (diagonal down), increasing or decreasing significantly (up or down), and/or whether they have crossed pre-defined thresholds in either direction (red or green coloring). Each arrow may be color-coded to indicate a corresponding range of values with regard to the corresponding information (FiO2 or PEEP), or to indicate a threshold has been reached. By providing an icon and color-based mapping of these patients, the subject technology provides the user with a simple and instantaneous view of the health of his/her patient population.

FIG. 10 is an example display screen indicating the health of ventilation-related equipment, according to various aspects of the subject technology. The depicted example includes visuals and data related to fleet management of multiple types of ventilators as well as pulmonary function testing equipment. The screen provides an image of the device 94 type along with the number of devices of that type which are online and offline and whether any messages exist for those devices. By providing a quick visual of this type, the invention gives biomedical staff or other users a broad view of what equipment needs attention, how the facility is operating and what specific actions need to be taken if any. By tapping on any of the equipment or device types, the user is taken to a visual screen which provides detailed information about individual devices as opposed to the entire population.

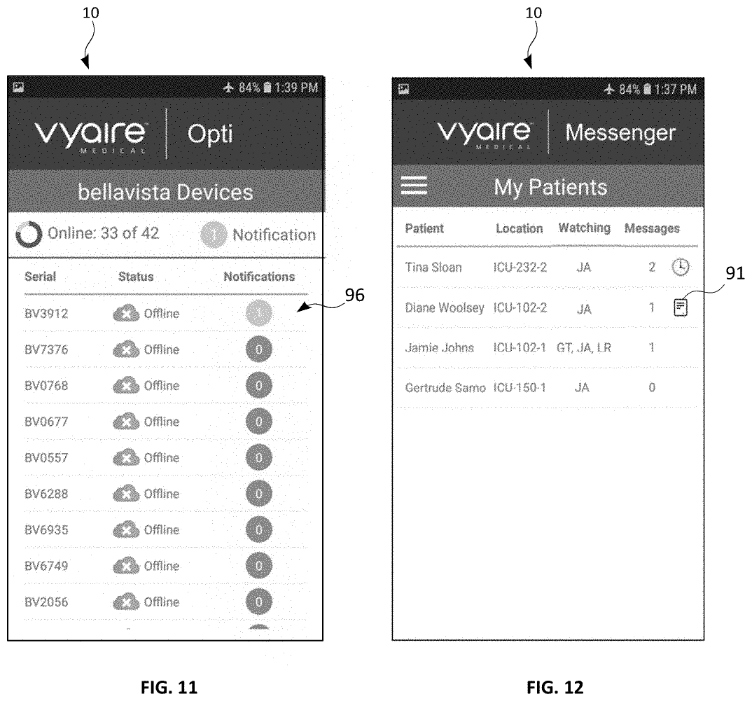

FIG. 11 depicts an example display screen indicating the status of a selected healthcare device, according to various aspects of the subject technology. Each device is represented in a line item detail 98 that includes the name of the device, a graphical indicator (e.g., a cloud) which visually depicts whether the device is connected to the system via network (e.g., “online”), and a number of notifications that the device generated. In the depicted example, a number of devices are offline, and that one of the devices has a notification or message. By tapping on the notification or message, the user can then see a display of the message which could, for example, be related to maintenance needs, data needs, connectivity issues, or any other functional issues that needs attention. Similar to patient messages, the system may push notifications or messages to users related to equipment. These messages are equally actionable and can also facilitate prioritization, coordination and cooperation of users as described in the clinical descriptions.

FIG. 12 depicts an example display screen indicating patients watched by the logged in user, with additional alert indicators for patients whose information includes a time-sensitive component or content, according to various aspects of the subject technology. In the depicted example, the user interface displays a clock icon next to a patient for whom a time-sensitive message or notification was received. Other icons may be displayed as well beside the message count to indicate other things. For example, it is useful for caregivers to be alerted to whether a new or active message relates to a Physician Order from the Computerized Provider Order Entry (CPOE) system. In this regard, FIG. 12 also depicts an example “order” icon 91 (next to patient “Diane Woolsey”) which indicates that an active message pertains to an active order.

In some implementations, the user interface may include code that, when a patient name is selected for a period of time (e.g., by touching and holding a finger on a patient name), the application may display additional pertinent information about the patient. In some embodiments, patient demographic information such as sex and age may be displayed. In some implementations, the system will cause the user interface to display which type of ventilation the patient is receiving—e.g. non-invasive, intubated, tracheal, how many days the patient has been on the ventilator, and scores related to patient condition such as APACHE II score and RASS. The APACHE II score is a calculated score determined from a number of physiologic parameters which predicts mortality at the beginning of admission to the ICU. The RASS is the Richmond Agitation-Sedation Score which is an indicator of the level of sedation and mood of a patient.

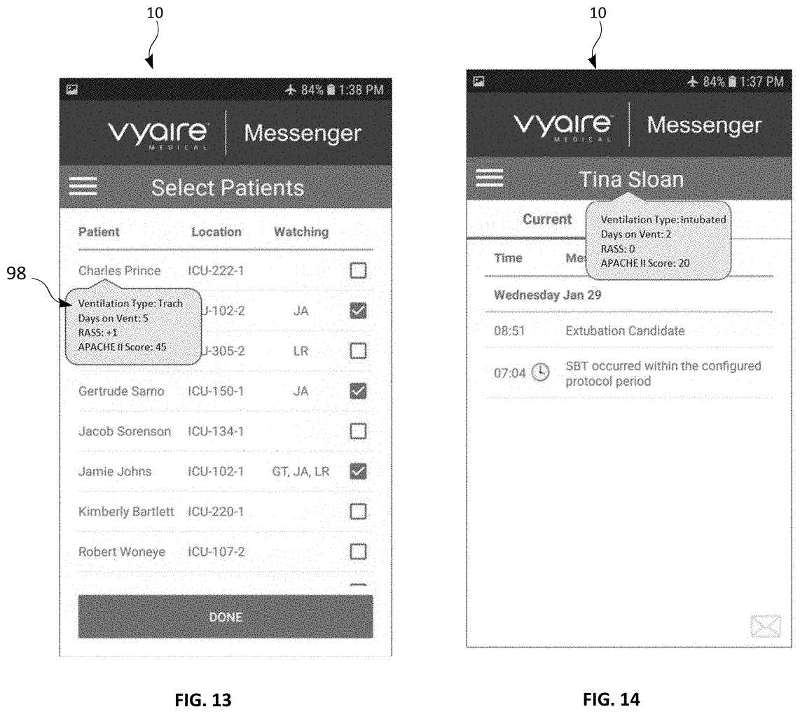

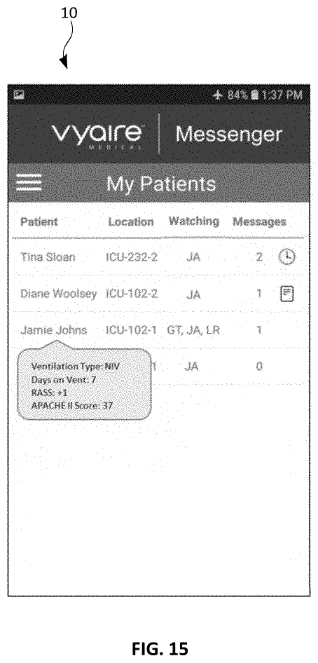

One or more of the previously described display screens may be configured to present a pop-out dialogue or window 98 displaying additional patient parameters. The patient pop-out information may be available on any application screen that displays a patient name for consistency and accessibility. In some implementations, a ventilation type may be displayed using an icon such as a mask, endotracheal tube, or tracheal icon. FIG. 13 depicts an example patient selection screen (as shown by FIG. 2), with patient information displayed in a pop-out dialog, according to various aspects of the subject technology. FIG. 14 depicts an example display screen for a selected patient, including a toggled layout design (as shown by FIG. 4), with patient information displayed in a pop-out dialog, according to various aspects of the subject technology. FIG. 15 depicts an example display screen indicating patients watched by the logged in user (as shown by FIG. 12), with patient information displayed in a pop-out dialog, according to various aspects of the subject technology.

In some embodiments, the application can display which ventilators are not associated to patients or send messages if a ventilator becomes disassociated from a patient. Knowing which patient type (e.g. tracheal vs. intubated) is important to the length of stay (LOS) calculations since tracheal patients are frequently ventilated for longer periods which can skew values for intubated patients. To this end, the system may calculate and display length of stay metrics (e.g., in FIG. 8), and may also take into account patient type for calculations, displays and messages. The messaging application may also send messages related to antibiotic use, sepsis tracking and other metrics related to infection prevention. Messages which are time sensitive and related to Ventilator Associated Events may contain countdown timer values—for example, “You have 13 hours left to address patient Y condition before it becomes a reportable event”. Length of stay metrics may be broken down, messaged and displayed based upon unit type such as Medical ICU, Surgical ICU, All ICUs, etc. Messages for clinicians related to intravenous drug administration may include notifications that medications are nearing empty or should be replaced within X minutes. Information related to patient conditions that is displayed during a touch motion may also include whether a patient is prone or supine. Proning a patient can affect certain ventilation parameters and may therefore necessitate adjustments in limits for alarm compliance and other ventilation strategies. Messages related to patient position may also be sent by the application.

Variants of the idea or invention comprise alternative ways to structure the visual indicators, alternative ways to provide messages or notifications (e.g. text or email), alternative sorting for prioritization, alternative icons for display of emphasis (e.g. time clock, arrows)

The subject technology enables a health organization and its clinicians to distill large amounts of data into simple and actionable insights delivered as push notifications and visuals within a mobile application for respiratory care and adjacent clinical insights. Messages, visuals, sorting and icons provide a clinician or user a simplified insight about their patients, facility or equipment. Messages, visuals sorting and icons enable prioritization of care, cooperation and coordination between adjacent clinical specialties and user profiles, and customization. Messages, visuals, sorting and icons filter noisy data environments into actionable insights. Consolidation and visual presentation of important health data such as length of stay and VAE surveillance—i.e. presentation of these as visual maps and/or scores enables clinicians and other health professionals to do their job efficiently, while reducing strain on medical environments by reducing the need for clinicians to repeatedly access shared computer devices or servers that service these healthcare devices.

FIG. 16 is a block diagram depicting a system for communicating health-related messages regarding ventilated patients, including an example ventilation device 102, ventilation management system 150, and home ventilation device 130, according to certain aspects of the subject technology. Ventilation management system 150 may include a server and, in many aspects, includes logic and instructions for providing the functionality previously described with regard to FIGS. 1 through 15. For example, a server of ventilation management system 150 may broker communications between the various devices, and/or generate user interface 10 for display by user devices 170. Ventilation device 102 and ventilation device 130 may represent each of multiple ventilation devices connected to ventilation management system 150. Although the ventilation management system 150 is illustrated as connected to a ventilation device 102 and a ventilation device 130, the ventilation management system 150 is configured to also connect to different medical devices, including infusion pumps, point of care vital signs monitors, and pulmonary diagnostics devices. In this regard, device 102 or device 130 may be representative of a different medical device.

Ventilation device 102 is connected to the ventilation management system 150 over the LAN 119 via respective communications modules 110 and 160 of the ventilation system 102 and the ventilation management system 150. The ventilation management system 150 is connected over WAN 120 to the home ventilation device 130 via respective communications modules 160 and 146 of the ventilation management system 150 and the home ventilation device 130. The ventilation device 130 is configured to operate substantially similar to the ventilation device 102 of the hospital 101, except that the ventilation device (or medical device) 130 is configured for use in the home 140. The communications modules 110, 160, and 146 are configured to interface with the networks to send and receive information, such as data, requests, responses, and commands to other devices on the networks. The communications modules 110, 160, and 146 can be, for example, modems, Ethernet cards, or WiFi component modules and devices.

The ventilation management system 150 includes a processor 154, the communications module 160, and a memory 152 that includes hospital data 156 and a ventilation management application 158. Although one ventilation device 102 is shown in FIG. 16, the ventilation management system 150 is configured to connect with and manage many ventilation devices 102, both ventilation devices 102 for hospitals 101 and ventilation devices 130 for use in the home 140.

In certain aspects, the ventilation management system 150 is configured to manage many ventilation devices 102 in the hospital 101 according to certain rules and procedures. For example, when powering on, a ventilation system 102 may send a handshake message to the ventilation management system 150 to establish a connection with the ventilation management system 150. Similarly, when powering down, the ventilation system 102 may send a power down message to the ventilation management system 150 so that the ventilation management system 150 ceases communication attempts with the ventilation system 102.

The ventilation management system 150 is configured to support a plurality of simultaneous connections to different ventilation devices 102 and ventilation devices 130, and to manage message distribution among the different devices, including to and from a user device 170. User device 170 may be a mobile device such as a laptop computer, tablet computer, or mobile phone. User device 170 may also be a desktop or terminal device authorized for use by a user. In this regard, user device 170 is configured with the previously described messaging application depicted by FIGS. 1 through 15 to receive messages, notifications, and other information from ventilation management system 150, as described throughout this disclosure.

The number of simultaneous connections can be configured by an administrator in order to accommodate network communication limitations (e.g., limited bandwidth availability). After the ventilation device 102 successfully handshakes with (e.g., connects to) the ventilation management system 150, the ventilation management system 150 may initiate communications to the ventilation device 102 when information becomes available, or at established intervals. The established intervals can be configured by a user so as to ensure that the ventilation device 102 does not exceed an established interval for communicating with the ventilation management system 150.

The ventilation management system 150 can receive or provide data to the ventilation device 102. For instance, alerts may be received from ventilation device 102 (or device 130) responsive to thresholds being exceeded. An admit-discharge-transfer communication can be sent to specified ventilation devices 102 within a certain care area of the hospital 101. Orders specific to a patient may be sent to a ventilation device 102 associated with the patient, and data specific to a patient may be received from ventilation device 102.

The ventilation device 102 may initiate a communication to the ventilation management system 150 if an alarm occurs on the ventilation system 102. The alarm may be indicated as time-sensitive and sent to the beginning of the queue for communicating data to the ventilation management system 150. All other data of the ventilation device 102 may be sent together at once, or a subset of the data can be sent at certain intervals.

Hospital data 156 may continuously or periodically received (in real time or near real time) by ventilation management system 150 from each ventilator device 102 and each ventilator device 130. The hospital data 156 may include configuration profiles configured to designate operating parameters for a respective ventilation device 102, operating parameters of each ventilation device 102 and/or physiological statistics or measurements of a patient associated with the ventilation device 102. Hospital data 156 also includes patient data for patients at the hospital 101, order (e.g., medication orders, respiratory therapy orders) data for patients at the hospital 101, and/or user data (e.g., for caregivers associated with the hospital 101).

The physiological statistics or measurements of the ventilator data includes, for example, a statistic(s) or measurement(s) indicating compliance of the lung (Cdyn, Cstat), flow resistance of the patient airways (Raw), inverse ratio ventilation (I/E), spontaneous ventilation rate, exhaled tidal volume (Vte), total lung ventilation per minute (Ve), peak expiratory flow rate (PEFR), peak inspiratory flow rate (PIFR), mean airway pressure, peak airway pressure, an average end-tidal expired CO2 and total ventilation rate. The operating parameters include, for example, a ventilation mode, a set mandatory tidal volume, positive end respiratory pressure (PEEP), an apnea interval, a bias flow, a breathing circuit compressible volume, a patient airway type (for example endotracheal tube, tracheostomy tube, face mask) and size, a fraction of inspired oxygen (FiO2), a breath cycle threshold, and a breath trigger threshold.

The processor 154 of the ventilation management system 150 is configured to execute instructions, such as instructions physically coded into the processor 154, instructions received from software (e.g., ventilation management application 158) in memory 152, or a combination of both. For example, the processor 154 of the ventilation management system 150 executes instructions to receive ventilator data from the ventilation device(s) 102 (e.g., including an initial configuration profile for the ventilation system 102).

Ventilation device 102 is configured to send ventilator information, notifications (or “alarms”), scalars, operating parameters 106 (or “settings”), physiological statistics (or “monitors”) of a patient associated with the ventilation device 102, and general information. The notifications include operational conditions of the ventilation device 102 that may require operator review and corrective action. Scalars include parameters that are typically updated periodically (e.g., every 500 ms) and can be represented graphically on a two-dimensional scale. The physiological statistics represent information that the ventilation device 102 is monitoring, and can dynamic based on a specific parameter. The operating parameters 106 represent the operational control values that the caregiver has accepted for the ventilation device 102. The general information can be information that is unique to the ventilation device 102, or that may relate to the patient (e.g., a patient identifier). The general information can include an identifier of the version and model of the ventilation device 102. It is also understood that the same or similar data may be communicated between ventilation management system 150 and ventilation device 130.

FIG. 16 further illustrates an example distributed server-client system for providing the disclosed user interface (represented by display screens of FIGS. 1 through 15). Ventilation management system 150 may include (among other equipment) a centralized server and at least one data source (e.g., a database 152). The centralized server and data source(s) may include multiple computing devices distributed over a local 119 or wide area network 120, or may be combined in a single device. Data may be stored in data source(s) 152 (e.g., a database) in real time and managed by the centralized server. In this regard, multiple medical devices 102, 130 may communicate patient data, over network 119, 120, to the centralized server in real time as the data is collected or measured from the patient, and the centralized server may store the patient data in data source(s) 152. According to some implementations, one or more servers may receive and store the patient data in multiple data sources.

According to various implementations, ventilation management system 150 (including centralized server) is configured to (by way of instructions) generate and provide virtual user interface 10 to clinician devices 170. In some implementations, ventilation management system 150 may function as a web server, and virtual interface 100 may rendered from a website provided by ventilation management system 150. According to various implementations, ventilation management system 150 may aggregate real time patient data and provide the data for display in virtual interface 100. The data and/or virtual interface 100 may be provided (e.g., transmitted) to each clinician device 170, and each clinician device 170 may include a software client program or other instructions configured to, when executed by one or more processors of the device, render and display virtual interface 100 with the corresponding data. The depicted clinician devices 170 may include personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity. While not shown in FIG. 16, it is understood that the connections between the various devices over local network 119 or wide area network 120 may be made via a wireless connection such as WiFi, BLUETOOTH, Radio Frequency, cellular, or other similar connection.

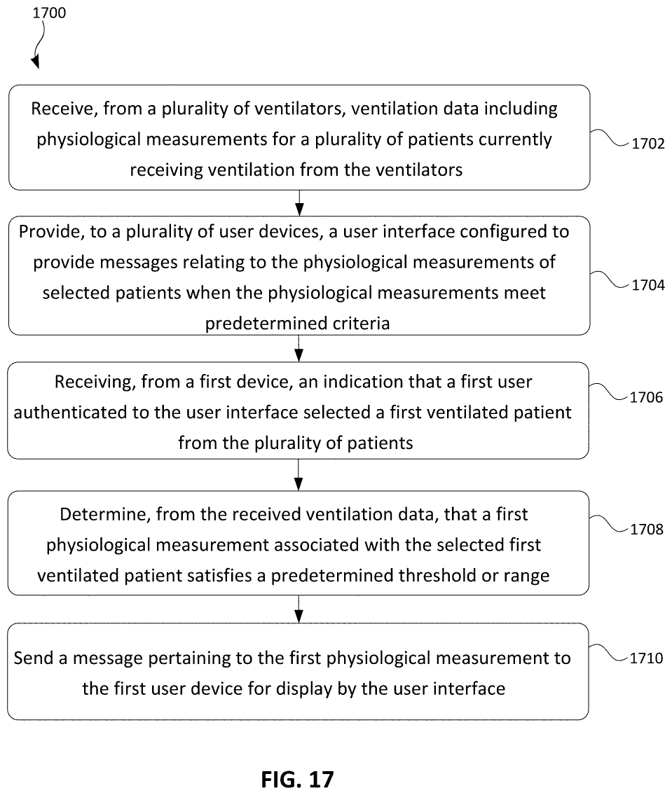

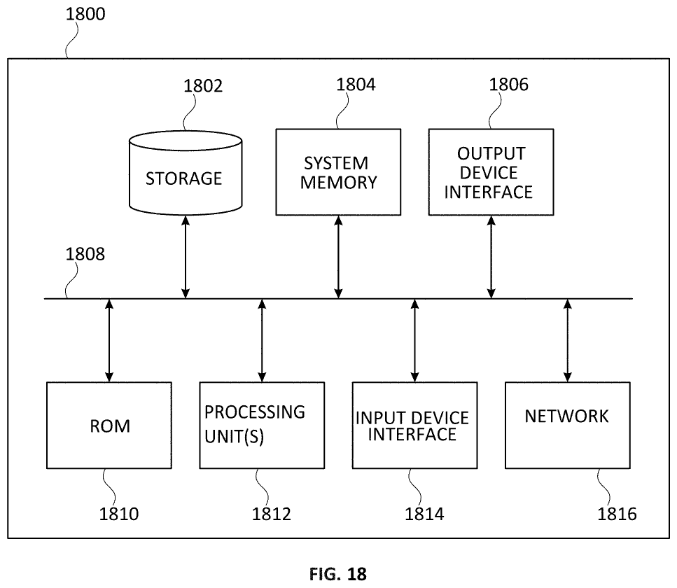

FIG. 17 depicts an example flow chart of a process 1700 of communicating health-related messages regarding ventilated patients, according to aspects of the subject technology. The process 1700 is implemented, in part, through the exchange of data between the ventilation device 102, the ventilation management system 150, and user device 170. For explanatory purposes, the various blocks of example process 1700 are described herein with reference to FIGS. 1 through 16, and the components and/or processes described herein. The one or more of the blocks of process 1700 may be implemented, for example, by a computing device, including a processor and other components utilized by the device. In some implementations, one or more of the blocks may be implemented apart from other blocks, and by one or more different processors or devices. Further for explanatory purposes, the blocks of example process 1700 are described as occurring in serial, or linearly. However, multiple blocks of example process 1700 may occur in parallel. In addition, the blocks of example process 1700 need not be performed in the order shown and/or one or more of the blocks of example process 1700 need not be performed.

In the depicted example flow diagram, ventilation data including physiological measurements (and, e.g., statistics) for a plurality of patients currently receiving ventilation is received from a plurality of ventilators (1702). A user interface 10 is provided to a plurality of user devices 170 (1704). The user interface 10 is configured to present the plurality of patients for selection by a respective user, receive from the respective user a selection of one or more selected patients of the plurality of patients, and provide, to the respective user, messages relating to physiological measurements of the selected patients when the physiological measurements of the selected patients meet predetermined criteria. The system 150 receives, from a first instance of the user interface 10 operating on a first user device 170 of the plurality of user devices, an indication that a first user authenticated to the user interface selected a first ventilated patient from the plurality of patients (1706). The system 150 determines, from the received ventilation data, that a first physiological measurement associated with the first ventilated patient satisfies a predetermined threshold or range of values (1708), and responsive to the first physiological measurement of the first ventilated patient satisfying the predetermined threshold or range of values, the system 150 sends a message to a the first user device for display by the first instance of the user interface when the first user is authenticated to the first user interface (1710), the message including information pertaining to the first physiological measurement.

Many aspects of the above-described example 1700, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described here is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 18 is a conceptual diagram illustrating an example electronic system 1800 for communicating health-related messages regarding ventilated patients, according to aspects of the subject technology. Electronic system 1800 may be a computing device for execution of software associated with one or more portions or steps of process 1800, or components and processes provided by FIGS. 1 through 17. Electronic system 1800 may be representative, in combination with the disclosure regarding FIGS. 1 through 17, of the ventilation management system 150 (or server of system 150) or the clinician device(s) 170 described above. In this regard, electronic system 1800 or computing device may be a personal computer or a mobile device such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Electronic system 1800 may include various types of computer readable media and interfaces for various other types of computer readable media. In the depicted example, electronic system 1700 includes a bus 1808, processing unit(s) 1812, a system memory 1804, a read-only memory (ROM) 1810, a permanent storage device 1802, an input device interface 1814, an output device interface 1806, and one or more network interfaces 1816. In some implementations, electronic system 1800 may include or be integrated with other computing devices or circuitry for operation of the various components and processes previously described.

Bus 1808 collectively represents all system, peripheral, and chipset buses that communicatively connect the numerous internal devices of electronic system 1700. For instance, bus 1808 communicatively connects processing unit(s) 1812 with ROM 1810, system memory 1804, and permanent storage device 1802.

From these various memory units, processing unit(s) 1812 retrieves instructions to execute and data to process in order to execute the processes of the subject disclosure. The processing unit(s) can be a single processor or a multi-core processor in different implementations.

ROM 1810 stores static data and instructions that are needed by processing unit(s) 1812 and other modules of the electronic system. Permanent storage device 1802, on the other hand, is a read-and-write memory device. This device is a non-volatile memory unit that stores instructions and data even when electronic system 1800 is off. Some implementations of the subject disclosure use a mass-storage device (such as a magnetic or optical disk and its corresponding disk drive) as permanent storage device 1802.

Other implementations use a removable storage device (such as a floppy disk, flash drive, and its corresponding disk drive) as permanent storage device 1802. Like permanent storage device 1802, system memory 1804 is a read-and-write memory device. However, unlike storage device 1802, system memory 1804 is a volatile read-and-write memory, such a random access memory. System memory 1804 stores some of the instructions and data that the processor needs at runtime. In some implementations, the processes of the subject disclosure are stored in system memory 1804, permanent storage device 1802, and/or ROM 1810. From these various memory units, processing unit(s) 1812 retrieves instructions to execute and data to process in order to execute the processes of some implementations.

Bus 1808 also connects to input and output device interfaces 1814 and 1806. Input device interface 1814 enables the user to communicate information and select commands to the electronic system. Input devices used with input device interface 1814 include, e.g., alphanumeric keyboards and pointing devices (also called “cursor control devices”). Output device interfaces 1806 enables, e.g., the display of images generated by the electronic system 1800. Output devices used with output device interface 1806 include, e.g., printers and display devices, such as cathode ray tubes (CRT) or liquid crystal displays (LCD). Some implementations include devices such as a touchscreen that functions as both input and output devices.

Also, as shown in FIG. 18, bus 1808 also couples electronic system 1700 to a network (not shown) through network interfaces 1816. Network interfaces 1816 may include, e.g., a wireless access point (e.g., Bluetooth or WiFi) or radio circuitry for connecting to a wireless access point. Network interfaces 1816 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 1700 can be used in conjunction with the subject disclosure.

These functions described above can be implemented in computer software, firmware or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (alternatively referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer,” “server,” “processor,” and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit this disclosure.

The term website, as used herein, may include any aspect of a website, including one or more web pages, one or more servers used to host or store web related content, etc. Accordingly, the term website may be used interchangeably with the terms web page and server. The predicate words “configured to,” “operable to,” and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

What is claimed is:

1. A method for communicating patient ventilation data, comprising:
receiving, from a plurality of ventilators, ventilation data including physiological measurements for a plurality of patients currently receiving ventilation from the plurality of ventilators;
providing, to one or more user devices, a user interface configured to present the plurality of patients for selection by a respective user, receive from the respective user a selection of one or more selected patients of the plurality of patients, and provide, to the respective user, messages relating to the physiological measurements of the selected patients when the physiological measurements of the selected patients meet predetermined criteria;
receiving, from the user interface operating on a first user device of the plurality of user devices, an indication that a first user authenticated to the user interface selected a first ventilated patient from the plurality of patients;
determining, from the received ventilation data, that a first physiological measurement associated with the selected first ventilated patient satisfies a predetermined threshold or range of values; and
responsive to the first physiological measurement of the first ventilated patient satisfying the predetermined threshold or range of values, sending a message pertaining to the first physiological measurement to the first user device for display by the user interface when the first user is authenticated to the user interface.

2. The method of claim 1, further comprising:
providing, based on the selection of the first ventilated patient by the first user, to the user interface operating on a second user device associated with a second user, an indication that the first user has selected to receive messages pertaining to the first ventilated patient.

3. The method of claim 1, wherein the plurality of patients are associated with a predetermined care area of a hospital, the method further comprising:
determining an average length of stay of the plurality of patients associated with the predetermined care area; and
providing, for display on the user interface, a graphical display of at least a portion of a circular dial having a plurality of color-coded regions, each region representative of a given range or threshold of time, the graphical display including a pointer overlapping the dial at a position representative of the determined average length of stay; and
providing, for display on the user interface, together with the graphical display of the dial, a bar graph having a plurality of bars, each bar representative of a historical average length of stay for a given prior period of time.

4. The method of claim 1, further comprising:
determining, for the first ventilated patient, from the ventilation data, a level of fraction of inspired oxygen (FiO2) and a level of positive-end expiratory pressure (PEEP); and
providing, for display by the user interface, a name of the first ventilated patient together with a plurality of directional arrows, each arrow indicative of a positive or negative or stable trend in the determined level of fraction of inspired oxygen (FiO2) or determined level of positive-end expiratory pressure (PEEP), and each arrow corresponding to a respective time period of a plurality of predetermined fixed time periods.

5. The method of claim 1, further comprising:
determining, for each of the plurality of ventilators, a status of the ventilator and a number of notifications associated with a patient receiving ventilation by the ventilator; and
providing, for display by the user interface, for each of the plurality of ventilators, an indication of the status of the ventilator and the number of notifications associated with the patient receiving ventilation by the ventilator.

6. A system comprising:
one or more processors;
a memory device containing instructions which, when executed by the one or more processors, cause the one or more processors to:
receive, from a plurality of ventilators, ventilation data including physiological measurements for a plurality of patients currently receiving ventilation from the plurality of ventilators;
provide, to a plurality of user devices, a user interface configured to present the plurality of patients for selection by a respective user, receive from the respective user a selection of one or more selected patients of the plurality of patients, and provide, to the respective user, messages relating to the physiological measurements of the selected patients when the physiological measurements of the selected patients meet predetermined criteria;
receive, from the user interface operating on a first user device of the plurality of user devices, an indication that a first user authenticated to the user interface selected a first ventilated patient from the plurality of patients;
determine, from the received ventilation data, that a first physiological measurement associated with the selected first ventilated patient satisfies a predetermined threshold or range of values; and
responsive to the first physiological measurement of the first ventilated patient satisfying the predetermined threshold or range of values, send a message pertaining to the first physiological measurement to the first user device for display by the user interface when the first user is authenticated to the user interface.

7. The system of claim 6, wherein the instructions, when executed, further cause the one or more processors to:
provide, based on the selection of the first ventilated patient by the first user, to the user interface operating on a second user device associated with a second user, an indication that the first user has selected to receive messages pertaining to the first ventilated patient.

8. The system of claim 6, wherein the plurality of patients are associated with a predetermined care area of a hospital, wherein the instructions, when executed, further cause the one or more processors to:
determine an average length of stay of the plurality of patients associated with the predetermined care area; and
provide, for display on the user interface, a graphical display of at least a portion of a circular dial having a plurality of color-coded regions, each region representative of a given range or threshold of time, the graphical display including a pointer overlapping the dial at a position representative of the determined average length of stay; and
provide, for display on the user interface, together with the graphical display of the dial, a bar graph having a plurality of bars, each bar representative of a historical average length of stay for a given prior period of time.

9. The system of claim 6, wherein the instructions, when executed, further cause the one or more processors to:
determine, for the first ventilated patient, from the ventilation data, a level of fraction of inspired oxygen (FiO2) and a level of positive-end expiratory pressure (PEEP); and
provide, for display by the user interface, a name of the first ventilated patient together with a plurality of directional arrows, each arrow indicative of a positive or negative or stable trend in the determined level of fraction of inspired oxygen (FiO2) or determined level of positive-end expiratory pressure (PEEP), and each arrow corresponding to a respective time period of a plurality of predetermined fixed time periods.

10. The system of claim 6, wherein the instructions, when executed, further cause the one or more processors to:
determine, for each of the plurality of ventilators, a status of the ventilator and a number of notifications associated with a patient receiving ventilation by the ventilator; and
provide, for display by the user interface, for each of the plurality of ventilators, an indication of the status of the ventilator and the number of notifications associated with the patient receiving ventilation by the ventilator.

11. A non-transitory computer-readable medium comprising instructions, which when executed by a computing device, cause the computing device to perform operations comprising:
receiving, from a plurality of ventilators, ventilation data including physiological measurements for a plurality of patients currently receiving ventilation from the plurality of ventilators;
providing, to a plurality of user devices, a user interface configured to present the plurality of patients for selection by a respective user, receive from the respective user a selection of one or more selected patients of the plurality of patients, and provide, to the respective user, messages relating to the physiological measurements of the selected patients when the physiological measurements of the selected patients meet predetermined criteria;
receiving, from the user interface operating on a first user device of the plurality of user devices, an indication that a first user authenticated to the user interface selected a first ventilated patient from the plurality of patients;
determining, from the received ventilation data, that a first physiological measurement associated with the selected first ventilated patient satisfies a predetermined threshold or range of values; and
responsive to the first physiological measurement of the first ventilated patient satisfying the predetermined threshold or range of values, sending a message pertaining to the first physiological measurement to the first user device for display by the user interface when the first user is authenticated to the user interface.

12. The non-transitory computer-readable medium of claim 11, the operations further comprising:
providing, based on the selection of the first ventilated patient by the first user, to the user interface operating on a second user device associated with a second user, an indication that the first user has selected to receive messages pertaining to the first ventilated patient.

13. The non-transitory computer-readable medium of claim 11, wherein the plurality of patients are associated with a predetermined care area of a hospital, the operations further comprising:
determining an average length of stay of the plurality of patients associated with the predetermined care area; and
providing, for display on the user interface, a graphical display of at least a portion of a circular dial having a plurality of color-coded regions, each region representative of a given range or threshold of time, the graphical display including a pointer overlapping the dial at a position representative of the determined average length of stay; and
providing, for display on the user interface, together with the graphical display of the dial, a bar graph having a plurality of bars, each bar representative of a historical average length of stay for a given prior period of time.

14. The non-transitory computer-readable medium of claim 11, the operations further comprising:
determining, for the first ventilated patient, from the ventilation data, a level of fraction of inspired oxygen (FiO2) and a level of positive-end expiratory pressure (PEEP); and
providing, for display by the user interface, a name of the first ventilated patient together with a plurality of directional arrows, each arrow indicative of a positive or negative or stable trend in the determined level of fraction of inspired oxygen (FiO2) or determined level of positive-end expiratory pressure (PEEP), and each arrow corresponding to a respective time period of a plurality of predetermined fixed time periods.

15. The non-transitory computer-readable medium of claim 11, the operations further comprising:
determining, for each of the plurality of ventilators, a status of the ventilator and a number of notifications associated with a patient receiving ventilation by the ventilator; and
providing, for display by the user interface, for each of the plurality of ventilators, an indication of the status of the ventilator and the number of notifications associated with the patient receiving ventilation by the ventilator.
